(19) United States
(12) Patent Application Publication
(10) Pub. No.: US 2010/0216160 A1
(43) Pub. Date: Aug. 26, 2010

(54) METHOD FOR THE SCREENING PROTEASOME OF ACTIVITY-MODULATING AGENTS AND MEANS FOR CARRYIING OUT SAID METHOD

(75) Inventors: Cécile Bougeret, Gif-Sur-Yvette (FR); Jean-Francois Briand, Orsay (FR); Patrick Zarzov, Viroflay (FR); Dominique Thomas, Gif-Sur-Yvette (FR)

Correspondence Address: YOUNG & THOMPSON, 209 Madison Street, Suite 500, Alexandria, VA, 22314, US

(73) Assignee: CYTOMICS SYSTEMS, Orsay (FR)

(21) Appl. No.: 12/090,867

(22) PCT Filed: Oct. 19, 2006
(86) PCT No.: PCT/FR06/51062

§ 371 Date: Jun. 9, 2008

(30) Foreign Application Priority Data
Oct. 19, 2005 (FR) 0553183

(51) Int. Cl. G01N 33/573; C12Q 1/66; C12Q 1/34; C12Q 1/02; C12N 1/19; C12N 15/63

(52) U.S. Cl. 435/7.4; 435/8; 435/18; 435/29; 435/254.2; 435/320.1

(57) ABSTRACT

An in cellulo method for the screening of proteasome activity-modulating agents, includes the following steps of:
a) bringing into contact the candidate agent with the yeast cell expressing in their nucleus a fusion protein comprising:
(i) a p21 polypeptide selected from the p21^{WAF1/Cip1} polypeptide and the p21[6KR]^{WAF1/Cip1} polypeptide; and
(ii) at least one detectable protein;
b) quantifying the first detectable protein in the yeast cells, at the end of at least one predetermined time interval after bringing into contact the candidate agent with the cells;
c) comparing the value obtained at the step (b) with a control value obtained when the step (a) is performed in the absence of the candidate agent.

### FIELD OF THE INVENTION

The field of the present invention relates to the screening of biologically active agents that are able to modulate the proteasome proteolytic activity, especially of therapeutically interesting agents intended to prevent or to treat cancers, inflammatory diseases, bacterial or fungal infections, muscular cachexia or neurodegenerative diseases.

### ANTERIOR ART

### The Ubiquitin-Proteasome Pathway

The concentration of any protein within human cells depends on the balance between the synthesis rate and the degradation rate thereof. The ubiquitin-proteasome pathway represents the main protein degradation mechanism in human cells. The ubiquitin-proteasome pathway-mediated protein degradation is a biological process that is very precisely regulated and controlled over the time. Such process, for a large majority of cases, does include two main successive steps: (i) the labeling of the proteins to be degraded by covalently adding an ubiquitin polymer and (ii) the destruction by the proteasome of the thus labeled proteins, which will be cleaved to small inactive peptides (Glickman and Ciechanover, 2002). A number of proteins are known, which are destroyed by the proteasome without being modified by adding an ubiquitin polymer. Likewise, peptide sequences have been identified that do lead to the degradation by the proteasome of the proteins containing the same, (like PATIO peptide unit, Hipp and al., 2005) without involving ubiquitin.

The ubiquitin-proteasome pathway does play a key role in very numerous biological processes. The proteasome-mediated protein degradation mechanisms indeed are involved in essential cellular mechanisms such as the DNA repair, the control of gene expression, the regulation of the cell cycle progression, the control of neosynthetized protein quality, the apoptosis or the immune response (Glickman and Ciechanover, 2002).

Dysfunctions of the ubiquitin-proteasome pathway that cause an abnormal degradation of the regulating proteins are known to be responsible for, or intimately involved in several genetic diseases and in many pathologies such as cancers (colorectal, lymphomas . . . ), inflammatory syndromes, neurodegenerative diseases such as Parkinson's disease or muscular atrophy (cachexia). The bypass of the ubiquitin-proteasome pathway by pat hogenic virus and bacteria (inducing the pathological degradation of human proteins) underlies a number of infection strategies.

The ubiquitin-proteasome field therefore provides a significant potential for developing new drugs.

### The Proteasome

Human cell proteasome is a very large-sized (>2.4 MDa) multiproteic complex, which is found in human cell cytoplasm and nucleus. Biochemical purifications of the proteasome that is present in human cells, as well as purifications of those proteasomes from other eukaryotic organism species, including yeasts, revealed that in all the eukaryotic organisms, the proteasome purified forms have two main sub-units, a proteolytic core, called the 20S proteasome, and a regulating complex, 195, which binds to each of both ends of the 20S proteasome (Coux and al., 1996; Glickman and Coux, 2001). The 205 proteasome is a particle which takes the form of a hollow cylinder, composed of 28 sub-units, distributed over 4 heptamer rings. Peptidase activities are present on the cylinder inner surface and influence each other in an allosteric way. There are three 205 proteasome-associated proteolytic activities (“trypsin-, chymotrypsin- and caspase-like”) which together contribute to degrade proteins to inactive peptides of from 3 to 20 amino acids long. In addition to the 205 proteasome, the 26S proteasome comprises the regulating complex 19S. This regulating complex 19S of 0.7 MDa comprises around 20 sub-units and may be partitioned in 2 main sub-domains, a “cover” required for recognizing the ubiquitinated proteins and a base which ensures the binding to the 20S proteasome. This base comprises 6 ATPases forming a ring assembly; ATP hydrolysis is indeed required, not only for the active degradation of the ubiquitinated proteins, but also for the unfolding and linearization steps for such proteins, which steps do occur prior to the proteolytic degradation of these proteins. Cap regulating sub-units are involved in multi-ubiquitinated protein recognition, while others take part to the deubiquitination stages that are required for unfolding the proteins to be degraded.

More recent studies using for instance immunopurification techniques have shown that the cellular proteasome comprises many proteins intimately associated with the 20S and 19S proteasomes thus highly suggesting that the cellular proteasome structural complexity is higher than that of the 26S proteasome. Such proteins include for example PA200 human proteins (which yeast homolog is the 240 kDa Blm10 protein (Schmidt and al., 2005), or PA28αβ or PA28αγ activators (Wojcik and al., 1998; Ustrell and al. 2002). The functional role of such proteins is not fully understood but said proteins are believed to ensure the ubiquitinated protein transfer to the proteasome, proteins regulating the ubiquitinated protein recognition by the proteasome or enzymes such as Hul5 (Leggett and al., 2002), which are involved in the polyubiquitin chain extension and would act to increase the transfer rate of the proteins to the peptidase sites. The cellular proteasome is believed to be responsible for the degradation of more than 80% of the cellular proteins.

### Proteasome and Cancer

It has been recently evidenced that the proteasome-mediated protein degradation represents a biological process which is essential for tumoral cell survival. Many studies did indeed clearly demonstrate that many types of malignant cells are more sensitive to proteasome inhibitors than normal cells. In vitro and in vivo assays conducted with proteasome inhibitors on various cancerous cell models have proven that this class of molecules does possess particular properties which make them attractive candidates for developing new therapeutic weapons against cancer. Lastly, inhibiting the proteasome activity does induce apoptosis and enhances the sensitivity of cancerous cells to traditional chemotherapies and radiotherapies. Likewise, it has been observed that inhibiting the proteasome substantially reduces the chemotherapy and radiotherapy resistance (Adams, 2004; Burger and Seth, 2004).

Inhibiting the degradation of regulators that are important for the cell cycle and homeostasis such as p21, p27 and p53 proteins has been involved as a mechanism through which the proteasome inhibition does affect the growth and the survival of tumour cells. Importantly, it has also been shown that inhibiting the proteasome blocks the activation of the cell regulator NF-κB which aberrant activation is a characteristic for a number of blood cancers. The characterization of proteasome inhibitors has revealed that the latter induce apoptosis (Pei and al., 2003), kill the tumour cells (Beverly and al. 1999), increase the sensitivity to radiations (Adams and Anderson, 2001) and allow for counteracting the drug resistance (Hideshima and al., 2001). In addition, it has been demonstrated that inhibiting the proteasome blocks the antiapoptotic signals that are elicited during radiotherapies or chemotherapies.

It should be also emphasized that the specific sensitivity of the malignant cells against proteasome inhibitors could result from their quick proliferation that is associated with dysfunctions of one or more checkpoint mechanism(s). As a consequence, these impairments do lead to a quick accumulation of very numerous defective proteins in malignant cells, which occurs in a much more strongly marked way as compared to normal cells. Such a quick and significant accumulation of mutant and/or incorrectly folded proteins are certainly likely to increase the dependence of malignant cells towards an active proteasome and therefore might make them highly sensitive to the proteasome inhibitors (Adams, 2004).

The proteasome inhibitors may be synthetic or natural ones. To date, five main families of inhibitors can be described: peptide aldehydes, peptide vinyl sulfones, peptide boron derivatives, peptide epoxy ketone derivatives and β-lactones. To date however, only two compounds have been developed up to the clinical stages after their anticancer properties were demonstrated; bortezomib (formerly PS-341) developed by the Millennium company (Cambridge, Mass., U.S.A.) and the NPI-0052 compound developed by the Nereus company (San Diego, Calif., U.S.A.)

Bortezomib, a boron derivative of a dipeptide, proved a real efficiency against a broad spectrum of cancer lines, including colorectal cancer lines, malignant lines derived from the central nervous system, prostate and breast cancer lines. Bortezomib is the first proteasome inhibitor that was tested in clinical trials. Positive results for Phase II and phase III trials, carried out on patients suffering from myelomas and for whom the two first treatments had been unsuccessful, demonstrated the efficiency of bortezomib and lead to the bortezomib marketing approval by the FDA in 2003 for treating myelomas resistant to traditional treatments. Bortezomib is marketed under the name Velcade®.

Bortezomib does very specifically inhibit one of the 3 proteolytic activities of the 20S proteasome, (the so called “chymotrypsin-like activity”). Its action triggers apoptosis and its effects seem to apply as much through the proapoptotic pathway activation as through the antiapoptotic pathway repression (Hideshima and al., 2001; Beverly and al., 1999).

The incidence of Velcade®-resistant cells and the fact that this compound because of its high toxicity has a limited application do generate a need for developing new proteasome inhibitors. In particular, as the known proteasome inhibitors are catalytic proteasome inhibitors, which may explain their toxicity, it would be interesting to be able to develop non catalytic proteasome inhibitors. Such non catalytic inhibitors could be active by inhibiting the activity of the 19S regulating complexes and the associated proteins which are necessary to the cellular proteasome to function (i.e., such as present in cells). Importantly, it should be noted that the known and developed inhibitors to date have been identified thanks to in vitro biochemical tests which report the purified proteasome proteolytic activity towards non natural substrates such as peptides freely diffusing within the 20S catalytic cylinder. Such tests thus do not report the cellular proteasome activity since they do not integrate for instance functions of the regulating complexes associated with the 205 proteasome. Generally speaking, there is therefore a need for identifying therapeutic compounds that are able to induce the destruction of the malignant cells.

### DESCRIPTION OF THE INVENTION

According to the invention, a method was developed for screening therapeutic agents of interest, which are selected for their selectivity of action on the cellular proteasome. This method for screening is carried out in cellulo and does enable, unlike existing biochemical tests, to identify catalytic and non catalytic inhibitors of cellular proteasome.

The applicant showed surprisingly that it is possible in yeast cells to mimic the degradation process, depending on the proteasome but not on a previous ubiquitination, of the human cell regulator p21^{WAF1/Cip1} by the proteasome, which is a naturally occurring process in human cells.

The p21^{WAF1/Cip1} protein is a regulator of the division cycle of human cells which belongs to the Cyclin-Dependent Kinases (Cdk) inhibitor family. It is the main function of the p21^{WAF1/Cip1} protein to regulate the activity of the Cdk2/cyclin complexes, the activation of which controls the cell cycle progression. The p21^{WAF1/Cip1} protein is also known as regulating the DNA synthesis by interacting with the PCNA factor (proliferating cell nuclear antigen factor), a sub-unit of DNA-polymerase δ crucial for the chromosomal replication. The p21^{WAF1/Cip1} protein is a very unstable, less structured protein (Kriwacki and al., 1996). There are two degradation pathways for the p21^{WAF1/Cip1} protein. The first one is ubiquitination-dependent and involves the SCF^{SkP2} ubiquitin ligase. This pathway is especially induced in UV-irradiated cells (Bendjennat and al. 2003; Bornstein and al., 2003). The second one does not require any ubiquitination of p21^{WAF1/Cip1} but is proteasome activity-dependent. This second degradation pathway which does not require any ubiquitination of p21^{WAF1/Cip1} seems to involve the Mdm2 protein in mammal cells, which protein would activate the degradation of p21^{WAF1/Cip1} by promoting its physical interaction with the proteasome (Zhang and al., 2004). The ubiquitin ligase function of Mdm2 is not involved in this mechanism.

Because the yeast cells do not express any orthologous protein or any protein similar to the Mdm2 human protein, it was absolutely unexpected for the applicant to show that it is yet possible to mimic, in yeast cells, the degradation of the p21^{WAF1/Cip1} human protein by the proteasome through a proteasome-dependent degradation pathway which does not require prior to degradation any ubiquitination stage of the p21^{WAF1/Cip1} protein.

It is an object of the present invention to provide an in cellulo method for the screening of proteasome activity-modulating agents, said method comprising the following steps of:
a) bringing into contact a candidate agent to be tested with recombinant yeast cells expressing in their nucleus a fusion protein comprising:
   (i) a p21 polypeptide selected from p21^{WAF1/Cip1} and p21[6KR]^{WAF1/Cip1} polypeptides; and
   (ii) at least one detectable protein;
b) quantifying said first detectable protein in the yeast cells, at the end of at least one predetermined time interval after bringing into contact the candidate agent with said cells;
c) in comparing the value obtained at the step (b) with a control value obtained when the step (a) is performed in the absence of the candidate agent.

The method of the invention consists in an in cellulo method given that the degradation of the p21^{WAF1/Cip1} or p21[6 KR]^{WAF1/Cip1} target protein is carried out and visualized in yeast cells and not by reactions carried out in an acellular system.

In some embodiments of the hereabove method for screening, yeast cells are used, which express a fusion protein comprising a mutant form of the p21^{WAF1/Cip1} protein wherein the 6 lysine residues of the p21 wild-type protein were substituted with arginine residues. This mutant form of the p21^{WAF1/Cip1} protein is referred to as p21[6KR]^{WAF1/Cip1}. These amino acid substitutions delete all the potential ubiquitination sites on the protein. The degradation of the p21[6KR]^{WAF1/Cip1} derivative, due to the lack of ubiquitination sites in the amino acid sequence thereof, does therefore exclusively and directly depend on the proteasome proteolytic activity and not on any ubiquitination stage.

At last, it has also been shown that in yeast cells the proteasome-mediated degradation of the p21 polypeptide (p21^{WAF1/Cip1} or p21[6KR]^{WAF1/Cip1}) does occur even when the p21 polypeptide (p21^{WAF1/Cip1} or p21[6KR]^{WAF1/Cip1}) is fused to a detectable protein and especially to an autofluorescent protein. Surprisingly, in the yeast cells, the fusion protein is 100% degraded, the polypeptides corresponding to the p21 protein (p21^{WAF1/Cip1} or p21[6KR]^{WAF1/Cip1}) and to the detectable protein are all degraded. These results are all the more surprising as in vitro biochemical experiments performed in the past demonstrated that when a fusion protein composed of p21^{WAF1/Cip1} fused to an autofluorescent protein is brought into contact with the 26S or 20S proteasome, then only the p21^{WAF1/Cip1} part is degraded, whereas the part corresponding to the autofluorescent part is released in a non degraded, stable form (Liu and al., 2003).

The entire degradation of the p21 fusion protein (p21^{WAF1/Cip1} or p21[6KR]^{WAF1/Cip1}), observed in the yeast cells, does enable to measure the fusion protein amount in the yeast cells by quantifying the signal generated by the detectable protein contained in said fusion protein.

The hereabove method does enable the man skilled in the art to determine whether a testable candidate agent may modify the degradation rate or the degradation degree of the p21 protein (p21^{WAF1/Cip1} or p21[6KR]^{WAF1/Cip1}) by the proteasome in the yeast cells expressing the human normal or mutated p21 protein (p21^{WAF1/Cip1} or p21[6KR]^{WAF1/Cip1}).

The hereabove in cellulo method for screening, as it does implement an artificial and humanized degradation system for a target fusion protein in yeast cells, does enable to screen agents which do specifically act on the cellular proteasome activity such as prevailing in all its complexity within a cell, and also in the absence of a previous ubiquitination stage of the target protein in the cell.

In addition, thanks to the hereabove mentioned method, a physiological test for screening agents that do act on the proteasome was developed, by creating in yeast a protein degradation metabolic pathway that does mimic the degradation pathway by the proteasome of the p21^{WAF1/Cip1} human regulating factor. Thus, as far as the protein degradation metabolic pathway is concerned, the method of the invention is carried out under physiological conditions that are very similar to the physiological conditions of a human proteasome-mediated protein degradation.

For the purposes of the present description, a proteasome activity “modulating agent” does include respectively, (i) an agent which does inhibit or block the proteasome activity, in particular which does inhibit or block the proteasome activity that does not depend on an ubiquitination step prior to degrading the target protein, and (ii) an agent which does activate or increase the proteasome activity, in particular which does activate or increase the proteasome activity that does not depend on an ubiquitination step prior to degrading the target protein.

Thanks to the hereabove mentioned method, agents that are able to inhibit the intracellular degradation rate or degree of the p21^{WAF1/Cip1} polypeptide or p21[6KR]^{WAF1/Cip1} polypeptide by the proteasome of the yeast cells may be identified. Those inhibitor agents which may be identified thanks to the method of the invention, since they will also inhibit the proteasome activity in human cells, are therapeutically interesting agents that may inhibit or block tumour cell proliferation, inhibit or block the expression activation of various genes involved in inflammation, autoimmune diseases or cancers, or disturb cell death control mechanisms (apoptosis).

Thus, the hereabove in cellulo screening method may comprise an additional step (d) consisting in positively selecting inhibitor candidate agents for which the amount of detectable protein as measured in step (b) is higher than the comparative control value.

The method of the invention makes also possible to identify agents that are able to increase the p21^{WAF1/Cip1} polypeptide or p21[6KR]^{WAF1/Cip1} polypeptide degradation rate or degree by the proteasome of the yeast cells. Such activating agents, because they will also activate the proteasome activity in human cells, are therapeutic agents of interest that may induce or increase the expression activation of various genes involved in inflammation, autoimmune diseases or cancers. Thus, according to this second aspect, the in cellulo screening method of the invention does enable to screen proinflammatory agents. Some of the proinflammatory agents selected in accordance with the present method may have therapeutic interest when they are used in small doses or when administered for a short period of time, for example as agents for inducing an early immune response, such as for inducing an infection-non specific resistance reaction or for activating antigen-presenting cells, for initiating an antigen-specific immune response, whether it is a humoral- or a cell-mediated response. Some other proinflammatory agents selected according to the in vitro screening method of the invention may consist in known active agents, for instance active agents for drugs, for which an undesirable proinflammatory effect is identified and for which special precautions for human health should be respected. Similarly, some agents selected in accordance with the present method may have proapoptotic activities.

Thus, according to another aspect, the screening method of the invention may comprise an additional step (d) consisting in positively selecting candidate activating agents, for which the amount of detectable protein as measured in step (b) is lower than the comparative control value.

As is well understood, the proteasome activity-modulating agent may be of any nature. Said agent may be any organic or mineral compound, and may be either a naturally occurring agent, or an agent prepared at least partially, by chemical or biological synthesis. Said agent may be in particular a peptide or a protein. Said agent also includes any molecule already known to possess a biological effect, especially a therapeutical effect or conversely a toxic effect proved or suspected for the organism.

In the method of the invention, once the fusion protein which contains the p21^{WAF1/Cip1} or p21[6KR]^{WAF1/Cip1} polypeptide fused to a detectable protein is expressed in the yeast cells, said fusion protein is recognized and does undergo a proteolysis which is performed by the proteasome. The quantification of the detectable protein contained in the yeast cell, at a given moment, makes it possible to determine the degradation degree of said fusion protein comprising the p21^{WAF1/Cip1} and the detectable protein or the p21[6KR]^{WAF1/Cip1} and the detectable protein, at this given moment.

A p21^{WAF1/Cip1} polypeptide of the invention consists of a polypeptide having at least 90% amino acid identity with the p21^{WAF1/Cip1} polypeptide of SEQ ID No 1. A p21^{WAF1/Cip1} polypeptide of the invention may be the p21^{WAF1/Cip1} polypeptide of SEQ ID No 1.

A p21[6KR]^{WAF1/Cip1} polypeptide of the invention consists of a polypeptide having at least 90% amino acid identity with the p21[6KR]^{WAF1/Cip1} polypeptide of SEQ ID No 2. A p21[6KR]^{WAF1/Cip1} polypeptide of the invention may consists in the p21[6KR]^{WAF1/Cip1} polypeptide of SEQ ID No 2.

For the purposes of the present description, a “nucleotide sequence” as used herein means either a polynucleotide or a nucleic acid. A “nucleotide sequence” does include the genetic material itself and thus is not limited to the only sequence listing.

A “nucleic acid”, a “polynucleotide”, an “oligonucleotide” or a “nucleotide sequence” do include RNA, DNA, cDNA or RNA/DNA hybrid sequences of more than one nucleotide, either in a single-strand or a double-strand configuration.

As used herein, a “nucleotide” means the natural nucleotides (A, T, G, C and U).

For the purposes of the present invention, a first polynucleotide is considered as being “complementary” to a second polynucleotide when each base of the first nucleotide is paired with the base complementary to the second polynucleotide which orientation is reversed. The complementary “bases” are A and T (or A and U), and C and G.

According to the invention, a first nucleic acid showing at least 90% identity with a second reference nucleic acid, will have at least 90%, preferably at least 91%, 92%, 93%, 94%, 95%, 96%, 97%, 97.5%, 98%, 98.3% 98.6%, 99%, 99.6% nucleotide identity with said second reference nucleic acid.

According to the invention, a first polypeptide showing at least 90% identity with a second reference polypeptide, will have at least 90%, preferably at least 91%, 92%, 93%, 94%, 95%, 96%, 97%, 97.5%, 98%, 98.3% 98.6%, 99%, 99.6% amino acid identity with said second reference polypeptide.

As used herein, the “identity percentage” between two nucleic acid sequences or between two polypeptide sequences is determined by comparing the two optimally aligned sequences through a comparative window.

The nucleotide sequence part or the amino acid sequence part in the comparative window may thus comprise additions or deletions (for example gaps) as related to the reference sequence (which does not include these additions or deletions) so as to obtain an optimal sequence alignment between the two sequences.

The identity percentage is calculated by determining the number of positions at which a same nucleic base or a same amino acid residue is observed for the two comparative sequences, then by dividing the number of positions at which there is an identity between the two nucleic bases or between the two amino acid residues, by the total number of the positions in the comparative window, then by multiplying the result by hundred so as to obtain the nucleotide identity percentage between the two sequences or the amino acid identity percentage between the two sequences.

The optimal sequence alignment for the comparison may be obtained with computers using known algorithms.

Most preferably, the sequence identity percentage is determined using the CLUSTAL W software (version 1.82) which parameters are set as follows: (1) CPU MODE=ClustalW mp; (2) ALIGNMENT=“full”; (3) OUTPUT FORMAT=“aln winumbers”; (4) OUTPUT ORDER=“aligned”; (5) COLOR ALIGNMENT=“no”; (6) KTUP (word size)=“default”; (7) WINDOW LENGTH=“default”; (8) SCORE TYPE=“percent”; (9) TOPDIAG=“default”; (10) PAIRGAP=“default”; (11) PHYLOGENETIC TREE/TREE TYPE=“none”; (12) MATRIX=“default”; (13) GAP OPEN=“default”; 14) END GAPS=“default”; (15) GAP EXTENSION=“default”; (16) GAP DISTANCES=“default”; (17) TREE TYPE “ciadogram” and (18) TREE GRAP DISTANCES=“hide”.

It was shown in accordance with the present invention that the sensitivity of the hereabove described screening method is enhanced when prior to bringing into contact the yeast cells with the candidate agent to be tested, the p21 target protein expression is stopped in the yeast cells, that is to say either the p21^{WAF1/Cip1}-detectable protein fusion protein, or the p21[6KR]^{WAF1/Cip1}-detectable protein fusion protein.

Thus, according to a first preferred embodiment of the hereabove method, the step (a) itself comprises the following steps of:
(a1) culturing the yeast cells which do express in their nucleus said fusion protein comprising the p21 polypeptide and at least one detectable protein;
(a2) stopping the expression by the yeast cells of said fusion protein comprising the p21 polypeptide and at least one detectable protein;
(a3) bringing into contact the yeast cells obtained at the end of the step (a2) with the candidate agent to be tested.

Stopping the expression of the p21^{WAF1/Cip1}-detectable protein fusion protein, at a chosen moment, may be easily performed by the man skilled in the art, by using to transform the yeast cells an expression cassette wherein the polynucleotide encoding said fusion protein is under the control of a promoter which is functional in the yeast cells and the activation of which, or conversely the repression of which, is induced by an inducing agent. Many inducible promoters, which are active in yeast cells, are known from the man skilled in the art, some of them being described hereafter in the description as well as in the examples.

In practice, the p21-detectable protein fusion protein is quickly recognized and degraded by the proteasome in the yeast cells.

The optimal conditions for implementing the screening method of the invention are conditions under which intracellular accumulation of the p21-detectable protein fusion protein expressed in the yeast cells is induced in a great amount. The greater the p21-detectable protein fusion protein intracellular amount in the beginning of the step a) of the method, the higher the value of the signal generated by the detectable protein, which may be very precisely measured during step b) of the method. Thus, the greatest the intracellular amount of the p21-detectable protein fusion protein at the beginning of the step a), the more accurately the variations in the fusion protein amount may be measured at the step b) of the method.

To accumulate a great intracellular amount of the p21-detectable protein fusion protein in the yeast cells, the sequence encoding said fusion protein is advantageously set under the control of a strong repressible promoter and yeast strains are used, which have several polynucleotide copies comprising (i) a strong repressible promoter, (ii) a nucleic acid encoding the p21-detectable protein fusion protein. The presence of a plurality of copies of the coding polynucleotide of interest does enable to obtain a strong detection signal of the detectable protein, at step a) of the method. These strong signal conditions do enable a very sensitive quantification of the detectable protein to be effected throughout the course of the method, since the p21-detectable protein fusion protein is being degraded by the proteasome. Obviously, the stronger the detectable initial signal, the more sensitive the measurements when implementing the method.

According to an advantageous embodiment of the screening method of the invention, yeast cells are used, in which the copy or copies of the polynucleotide encoding the p21-detectable protein fusion protein is or are integrated into the chromosome. This advantageous embodiment does enable to intracellularly accumulate the p21-detectable protein fusion protein to a high level in all the yeast cells cultured for implementing the present method. This embodiment of the screening method of the invention is advantageous as compared to using plasmid-transformed yeast cells in each of which one or more copy or copies of the polynucleotide encoding the p21-detectable protein fusion protein is or are inserted. Indeed, when the yeast cells expressing the p21-detectable protein fusion protein consist in cells comprising one or more recombinant vector(s), in particular one or more recombinant plasmids, wherein at least one copy of the polynucleotide encoding the p21-detectable protein fusion protein is inserted, a non-homogeneous transmission of said interesting recombinant vectors may occur, with the successive yeast cell generations. In some cases, the intracellular expression level of the p21-detectable protein fusion protein would be heterogeneous within all of the cultured yeast cells, which could reduce the sensitivity of the screening method of the invention.

### Description of the Screening Method Preferred Embodiments

The preferred embodiments of the screening method of the invention are described hereafter, especially as related to the description of the functional and structural aspects of the various means for carrying out said method.

Generally speaking, the detectable protein which is contained in the p21-detectable protein fusion protein may be of any nature, provided that its presence can be specifically detected in the yeast cells prior to proteolysis thereof, and that the presence of detectable protein proteolyzed forms, especially of peptide fragments resulting from the proteolysis of said detectable protein, are not detected by the specific detection means that is selected.

As will be easily understood, the proteasome proteolytic activity is monitored according to the method of the invention by measuring the effect thereof on the p21-detectable protein fusion protein stability. By expressing in the yeast cells the p21 factor in the form of a fusion protein, the degradation of the p21-containing fusion protein may be followed in real time by detecting the non proteolized detectable protein.

Preferably, the p21-detectable protein fusion protein comprises a mutant form of the p21^{WAF1/Cip1} polypeptide wherein the 6 lysine residues of the p21 protein were substituted with arginine residues, which is referred to as p21[6KR]^{WAF1/Cip1}. The p21[6KR]^{WAF1/Cip1}-detectable protein fusion protein stability depends exclusively on the proteasome proteolytic activity and does not involve any previous ubiquitination thereof. Depending on the nature of the p21-fused detectable protein (p21^{WAF1/Cip1} or p21[6KR]^{WAF1/Cip1}), the degradation of the fusion protein may be monitored by means known per se, for instance by fluorescence measuring systems using either a flow cytometer, a microplate reader, a fluorimeter, or a fluorescent microscope, or through colorimetric, enzymatic or immunological techniques. As an illustration, the detectable protein may be selected from an antigen, a fluorescent protein or a protein having an enzymatic activity.

When the detectable protein consists in an antigen, it may be any type of antigen, provided that antibodies specifically directed against this antigen are already available or alternatively may be prepared in accordance with any method suitable for preparing antibodies, especially polyclonal or monoclonal antibodies, well known from the man skilled in the art. Preferably, in this case, the detectable protein is a small-sized antigen which may not interfere with the p21 protein (p21^{WAF1/Cip1} or p21[6KR]^{WAF1/Cip1}) recognition by the proteasome. Thus, preferably, a 7 to 100 amino acid-long chain peptide will be used as an antigen, more preferably a 7 to 50 amino acid-long chain peptide, and even more preferably a 7 to 30 amino acid-long chain peptide, for example a 10 amino acid-long chain peptide. As an illustration, the HA antigen of sequence [NH₂-YPYDVPDYA-COOH] SEQ ID No 7, or a FLAG antigen of sequence [NH₂-DYKDDDDK-COOH] SEQ ID No 8 (FLAG monomer) or of sequence [NH₂-MDYKDHDGDYKDHOIDYKDDDDIK-COOH] SEQ ID No 9 (FLAG trimer). In this case, to quantify the detectable protein at the step (b) of the method, an antibody is used which specifically recognizes the antigen contained in the fusion protein, this antibody being directly or indirectly labeled. The quantification is then performed by measuring the detectable signal produced by the complexes that were formed in the yeast cells between the labeled antibody and the p21-antigen fusion protein. Thus, at the step (b), when the first detectable protein consists in an antigen, said first detectable protein is quantified by detecting the complexes formed between said protein and antibodies that recognize the same.

When the detectable protein is an intrinsic fluorescence protein, it is especially selected from the mAG protein, the GFP protein or a derivative thereof, the YFP protein or a derivative thereof, and the dsRED protein. Amongst the proteins derived from the GFP protein, any of the proteins known under the name GFPMut3 (Cormack and al. (Gene (1996) 173: 33-38)), Venus (Nagai and al., (Nat. Biotechnol. (2002) 20:87-90)) or Sapphire (Zapata-Hammer and Griesbeck, BMC Biotechnol. (2003) 3:5) may be used.

The intrinsic fluorescence protein may also be selected from autofluorescent proteins derived from various organisms, other than Aequorea victoria. The intrinsic fluorescence protein may be for instance selected from following proteins:
the CopGFP protein derived from Pontellina plumate, and described by D. A. Shagin and al. (2004, Mol. Biol. Evol. 21:841-850);
the TurboGFP protein, a CopGFP variant; and described by D. A. Shagin and al., 2004 (Mot. Biol. Evol, 21:841-850);
the PhiYFP protein derived from Phialidium sp.; and described by D. A. Shagin and al. (2004, Mol. Biol. Evol. 21:841-850);
the AcGFP protein derived from Aequorea coeruiescens, as well as variants thereof, and described by N. G. Gurskaya, (2003, Biochem. J. 373:403-408); and
the dsRED protein derived from Discosoma sp.; and described by M. V. Matz and al (1999, Nature Biotech. 17:969-973).

Preferably the mAG protein will be used in the p21-detectable protein fusion protein, which is also called “monomeric Azami-Green”, and is derived from Galaxeidae coral; and described by Karasawa and al. (Karasawa and al., J. Biol. Chem. 2003 278:34167-34171).

When the detectable protein consists in an intrinsic fluorescence protein, the detectable protein is quantified at the step (b) of the method by measuring the fluorescence signal emitted by the p21-fluorescent protein fusion protein using any suitable device. Thus, at the step (b), when the detectable protein is a fluorescent protein, said detectable protein is quantified by measuring the fluorescence signal emitted by said protein.

When the detectable protein consists in a protein having an enzymatic activity, said detectable protein is selected for instance from luciferase and β-lactamase. In this case, the detectable protein is quantified at the step (b) of the method by measuring the amount of the product(s) resulting from the enzyme-mediated substrate transformation. When the product of the enzymatic activity is coloured, the measurement may be effected by colorimetry. When the product of the enzymatic activity is fluorescent, the intensity of the fluorescence signal emitted by said product is measured by means of any suitable fluorescence measuring device. Thus, at the step (b), when the first detectable protein is a protein having an enzymatic activity, said detectable protein is quantified by measuring the amount of the substrate that was transformed by said protein.

According to a preferred embodiment, the p21^{WAF1/Cip1} polypeptide-containing protein is the protein with the amino acid sequence of SEQ ID No 3, which may be encoded by the nucleic acid of SEQ ID No 5. The protein of SEQ ID No 3 contains, from the NH₂-terminal end to the COON-terminal end, respectively (i) the detectable mAG protein sequence beginning from amino acid at position 1 to amino acid at position 226, (ii) a first spacer peptide beginning from amino acid at position 227 to amino acid at position 228 and (iii) the p21^{WAF1/Cip1} polypeptide beginning from amino acid at position 229 to amino acid at position 392. The nucleic acid of SEQ ID No 5 contains, from the 5′ end to the 3′ end, respectively (i) the sequence encoding the detectable mAG protein beginning from the nucleotide at position 1 to the nucleotide at position 678, (ii) the sequence encoding a spacer peptide beginning from the nucleotide at position 679 to the nucleotide at position 684 and (iii) the sequence encoding the p21^{WAF1/Cip1} polypeptide beginning from the nucleotide at position 685 to the nucleotide at position 1179 (codon stop included).

According to another preferred embodiment, the p21[6KR]^{WAF1/Cip1} polypeptide-containing protein is the protein of SEQ ID No 4, which may be encoded by the nucleic acid of SEQ ID No 6. The protein of SEQ ID No 4 contains, from the NH₂-terminal end to the COON-terminal end, respectively (i) the detectable mAG protein sequence beginning from amino acid at position 1 to amino acid at position 226, (ii) a first spacer peptide beginning from amino acid at position 227 to amino acid at position 228 and (iii) the p21[6KR]^{WAF1/Cip1} polypeptide beginning from amino acid at position 229 to amino acid at position 392. The nucleic acid of SEQ ID No 6 contains, from the 5′ end to the 3′ end, respectively (i) the sequence encoding the detectable mAG protein beginning from the nucleotide at position 1 to the nucleotide at position 678, (ii) the sequence encoding a spacer peptide beginning from the nucleotide at position 679 to the nucleotide at position 684 and (iii) the sequence encoding the p21[6KR]^{WAF1/Cip1} polypeptide beginning from the nucleotide at position 685 to the nucleotide at position 1179 (codon stop included).

In polynucleotides of SEQ ID No 5 encoding the p21^{WAF1/Cip1}-containing fusion protein and of SEQ ID No 6 encoding the p21[6KR]^{WAF1/Cip1}-containing fusion protein, the identity of the nucleic bases was adapted so as to include within these nucleic acid sequences, the codons which are preferably used in the yeast cells.

According to a further aspect, the screening method of the invention is characterized in that the recombinant yeast cells are transformed with a polynucleotide which comprises
(a) an open reading frame encoding fusion protein comprising (i) the p21 polypeptide (p21^{WAF1/Cip1} or p21[6KR]^{WAF1/Cip1}) and (ii) a detectable protein, and
(b) a regulating sequence which is functional in yeast cells and controls the expression of said open reading frame.

The hereabove polynucleotide may be the nucleic acid of SEQ ID No 5 or the nucleic acid of SEQ ID No 6.

### Preferred Nucleic Acids, Expression Vectors and Transformed Yeast Cells According to the Present Invention.

According to the invention, nucleic acids were synthesized which, when introduced in yeast cells, do induce the expression of the p21-detectable protein fusion protein, that is to say nucleic acids encoding the p21^{WAF1/Cip1}-detectable protein fusion protein and nucleic acids encoding the p21[6KR]^{WAF1/Cip1}-detectable protein fusion protein.

Each of the synthesized nucleic acids comprises a coding sequence, which is also referred to as an “open reading frame” or “ORF” which encodes the interesting p21-detectable protein fusion protein. Illustrative examples of the nucleic acids of the invention include nucleic acids of SEQ ID No 5(p21^{WAF1/Cip1}-detectable protein) and SEQ ID No 6 (p21[6KR]^{WAF1/Cip1}-detectable protein), which structure was previously described in the description.

Each of the nucleic acids also comprises a regulating sequence comprising a promoter which is functional in the yeast cells.

In a preferred embodiment, the promoter which is functional in the yeast cells is a repressible promoter, that is to say a promoter which is functional in the yeast cells and sensitive to the action of an inducing agent. When the inducing agent is added to the yeast cell culture medium, it does induce the repression or the inhibition of the expression of the sequence encoding the interesting protein under the control thereof. Such repressible promoter is advantageously selected from CUP1, GAL1, GAL10, MET3, MET25, PHO5, PHO87 and TH14 promoters derived from the yeast Saccharomyces cerevisiae.

The sequence of the GAL1 gene promoter from the yeast S. cerevisiae that may be used according to the invention includes the one described by Johnston and Davis (Mol. Cell. Biol. (1984) 4: 1440-1448).

The sequence of the MET3 gene promoter from the yeast S. cerevisiae that may be used according to the invention includes the one described by Cherest and al. (Mol. Gen. Genet. (1987) 210 (2): 307-313). The sequence of the MET25 gene promoter from the yeast S. cerevisiae that may be used according to the invention includes the one described in Kerjan and al. (Nucleic Acids Res. (1986) 14(20): 7861-7871).

The sequence of the PHO5 gene promoter from the yeast S. cerevisiae that may be used according to the invention includes the one described by Feldmann and al. (EMBO J. (1994) 13(24): 5795-5809).

The sequence of the TH14 gene promoter from the yeast S. cerevisiae that may be used according to the invention includes the one described by Skala and al., Yeast (1995) 14:1421-1427.

The sequence of the CUP1 gene promoter from the yeast S. cerevisiae that may be used according to the invention includes the one described by Karin and al. (PNAS (1984) 81(2): 337-341).

In a preferred embodiment, the nucleic acid or the polynucleotide which encodes the p21-detectable protein fusion protein comprises the GAL1 regulating sequence. This sequence activates the expression of the open reading frame encoding the p21 polypeptide-containing fusion protein when the yeast cells are cultured in the presence of galactose. On the contrary, this sequence represses the expression of the open reading frame encoding the p21 polypeptide-containing fusion protein when the yeast cells are cultured in the presence of glucose.

Thus, in an advantageous embodiment of the screening method of the invention, the expression of the p21 polypeptide-containing fusion protein is carried out temporarily during the screening test. After having been induced for a predetermined time period that may range from 20 minutes to 24 hours, the expression of the p21-containing protein is specifically stopped (in an experiment known from the man skilled in the art under the name “promoter shut off”) prior to exposing the cells to the molecules to be screened. Such stopping of the expression is obtained by adding to the culture medium a molecule that is able to repress the activity of the promoter controlling the expression of the p21-detectable protein fusion protein, or by removing in the culture medium an agent or a molecule that is required for activating said promoter.

Thus, when the p21-detectable protein fusion protein is expressed under the control of the GAL1 gene promoter, then the expression of this promoter is repressed by adding glucose to the final concentration of 2% in the culture medium. Stopping the neosynthesis of the p21-containing fusion protein makes it possible to measure the stability thereof in realtime by determining for example the yeast cell fluorescence over time after synthesis Shutt off, in the embodiment wherein said fusion protein contains a detectable intrinsic fluorescence protein, such as mAG or a GFP-derived protein.

Thus, it is also an object of the present invention to provide an expression cassette which is functional in the yeast cells comprising a polynucleotide having an open reading frame encoding the fusion protein comprising the p21 polypeptide and at least one detectable protein, and a regulating sequence which is functional in yeast cells and controls the expression of said open reading frame.

Such an expression cassette may be characterized in that the p21 polypeptide is selected from the p21^{WAF1/Cip1} polypeptide and the p21[6KR]^{WAF1/Cip1} polypeptide.

Such an expression cassette may especially be characterized in that the p21 polypeptide is selected from the p21^{WAF1/Cip1} polypeptide of SEQ ID No 1 and the p21[6KR]^{WAF1/Cip1} polypeptide of SEQ ID No 2.

Such an expression cassette may especially comprise or consist in the nucleic acid of SEQ ID No 5 of the invention, which encodes the mAG-p21^{WAF1/Cip1} fusion protein of SEQ ID No 3.

Such an expression cassette may also comprise or consist in the nucleic acid of SEQ ID No 6 of the invention, which encodes the mAG-p21[6KR]^{WAF1/Cip1} fusion protein of SEQ ID No 4.

According to a preferred embodiment of such an expression cassette, the regulating sequence comprises a repressible promoter, which is functional in the yeast cells, and sensitive to the action of an inducing agent, such as a promoter selected from the CUP1, GAL1, GAL10, MET3, MET25, PHO5, and THI4 promoters from the yeast Saccharomyces cerevisiae.

According to a further advantageous embodiment of the screening method of the invention, the recombinant yeast cells do comprise the nucleic acid or the polynucleotide comprising the sequence encoding the p21-detectable protein fusion protein in a form that is integrated in the genome thereof, as illustrated in the examples.

In yet another embodiment of the screening method of the invention, the yeast cells are transformed by the nucleic acid or the polynucleotide comprising the sequence encoding the p21-detectable protein fusion protein which is in a form that is non integrated to the chromosomes, for example in the form of vectors which are functional in the yeast cells and which carry at least one replication origin which is functional in the yeast cells.

Generally speaking, for implementing the screening method of the invention, yeast cells will be advantageously used, which possess a good membrane permeability, especially a good membrane permeability towards the agents to be tested with the present method.

For implementing the preferred embodiment of the screening method of the invention wherein the expression of the fusion protein is carried out under the control of a repressible promoter, yeast cells will be also advantageously used, which possess a good membrane permeability towards the “inducing” compounds to which said repressible promoters are sensitive.

Thus, according to another preferred embodiment of the screening method of the invention, yeast strains will be used which genome comprises one or more mutation(s) increasing the permeability towards the agents to be tested, such as mutations inactivating the PDR1 and PDR3 genes, two genes encoding transcription factors which in the yeast control the expression of transporters inserted into the plasma membrane (Vidal and al., 1999, Nourani and al., 1997).

Yeast strains will be preferably used, which possess the genetic background of the W303 strain of the yeast Saccharomyces cerevisiae described by Bailis and al. (1990), or any other characterized strain of said yeast Saccharomyces cerevisiae.

The transformation of the yeast cells by exogenous DNA is preferably performed by using methods known from the man skilled in the art, especially the method described by Schiestl and al. (1989). The various yeast strains were prepared by using genetic methods (breeding, sporulation, ascus dissection and spore phenotypic analysis) known and especially described by Sherman and al. (1979) and the methods of reverse genetics described for instance by Rothstein (1991).

According to the invention, yeasts are preferably transformed by plasmids constructed using traditional molecular biology techniques, for instance the protocols described by Sambrook and al. (1989) and Ausubel and al. (1990-2004).

Thus, it is a further object of the invention to provide an expression vector characterized in that it comprises an expression cassette such as defined in the present description.

A first vector according to the invention is the pCSYAQ6-p21 wt vector which is described in the examples, and which contributed to prepare the CYS343 yeast strain.

A second vector according to the invention is the pCSYAQ6-p21[6KR] vector which is described in the examples, and which contributed to prepare the CYS344 yeast strain.

The present invention also relates to a recombinant yeast strain comprising, in a form integrated in the genome thereof, a polynucleotide which comprises (a) an open reading frame encoding the fusion protein comprising a p21 polypeptide (p21^{WAF1/Cip1} or p21[6KR]^{WAF1/Cip1}) and at least one detectable protein, and (b) a regulating sequence which is functional in yeast cells and controls the expression of said open reading frame.

A recombinant yeast strain according to the invention may consist in a recombinant yeast strain comprising, in a form integrated in the genome thereof, a polynucleotide which comprises (a) an open reading frame encoding the fusion protein comprising a p21^{WAF1/Cip1} polypeptide of SEQ ID No 1 and at least one detectable protein, and (b) a regulating sequence which is functional in yeast cells and controls the expression of said open reading frame.

A recombinant yeast strain according to the invention may consist in a recombinant yeast strain comprising, in a form integrated in the genome thereof, a polynucleotide which comprises (a) an open reading frame encoding the fusion protein comprising a p21[6KR]^{WAF1/Cip1} polypeptide of SEQ ID No 2 and at least one detectable protein, and (b) a regulating sequence which is functional in yeast cells and controls the expression of said open reading frame.

In particular, the present invention relates to a recombinant yeast strain such as defined hereabove, which consists in the CYS343 yeast strain, which expresses the fusion protein comprising the p21^{WAF1/Cip1} polypeptide and the detectable mAG protein, that is to say the fusion protein of SEQ ID No 3.

In particular, the present invention relates to a recombinant yeast strain such as defined hereabove, which consists in the CYS344 yeast strain, which expresses the fusion protein comprising the p21[6KR]^{WAF1/Cip1} polypeptide and the detectable mAG protein, that is to say the fusion protein of SEQ ID No 4.

The present invention also relates to a kit for screening proteasome activity-modulating agents, characterized in that it comprises an expression vector comprising an expression cassette encoding the fusion protein containing a p21 polypeptide (p21^{WAF1/Cip1} or p21[6KR]^{WAF1/Cip1}) such as defined hereabove.

The present invention also relates to a kit for screening proteasome activity-modulating agents characterized in that it comprises recombinant yeast cells comprising, in a form integrated in the genome thereof, an expression cassette encoding the fusion protein containing a p21 polypeptide (p21^{WAF1/Cip1} or p21[6KR]^{WAF1/Cip1}) such as defined hereabove.

Preferably, the hereabove kit comprises recombinant yeast cells of CYS343 yeast strain or recombinant yeast cells of CYS344 yeast strain.

The screening method of the invention does enable to visualise the proteasome activity towards a human target protein in yeast cells, and more precisely towards a p21 target protein, that is to say either the target protein consisting in a fusion protein comprising the p21^{WAF1/Cip1} human protein fused to a detectable protein, or the p21[6KR]^{WAF1/Cip1} mutated protein fused to a detectable protein.

This method is particularly advantageous for screening molecules or agents that are able to act on diseases associated with an excessive or an insufficient degradation of the cellular proteins, such as some cancers, inflammatory and immune syndromes, fungal, bacterial and viral infections or some diseases of the central nervous system.

The main advantages of the screening method of the invention include especially the following ones:
easiness of implementation: the proteasome activity as present in the cells is visualized very simply thanks to the controlled expression of the p21^{WAF1/Cip1} wild-type or mutant human factor in the yeast cells. In addition, when the p21^{WAF1/Cip1} factor is expressed as a hybrid protein fused with an intrinsic fluorescence protein, such as mAG or GFP, the proteasome activity towards the p21^{WAF1/Cip1} factor or the p21[6KR]^{WAF1/Cip1} factor is directly measured by quantifying the fluorescence emitted by the hybrid protein. Likewise, when the p21^{WAF1/Cip1} factor or the p21[6KR]^{WAF1/Cip1} factor is expressed as a hybrid protein fused with a protein such as luciferase, the proteasome activity towards the p21^{WAF1/Cip1} factor or the p21[6KR]^{WAF1/Cip1} factor is directly measured by quantifying the luminescence emitted by the hybrid protein in the presence of a substrate such as fluorescein.
Adequacy with the therapeutic context: the proteasome activity is followed by performing a functional assay made in entire cells. The in cellulo screening method of the invention does therefore enable to select molecules that are able to activate or inhibit the proteasome activity in a context similar to that of the final therapeutic use.
specificity; although it is performed in cellulo in the cell, the screening method of the invention is specific as it relies on the expression, in an organism heterologous as related to the human body, of the p21 human protein. The molecules that are selected thanks to the screening method of the invention will be specific for the proteasome activity, and thus will not be molecules selected for example for their ability to interfere with one of the numerous signalling pathways inducing the p21^{WAF1/Cip1} factor degradation in the human cells. Indeed, when implementing the screening method of the invention, the degradation of the p21^{WAF1/Cip1} or p21[6KR]^{WAF1/Cip1} by the proteasome is induced by a fully artificial and absolutely reproducible metabolic pathway, as for example, by adding glucose to block the GAL1 promoter activity when p21^{WAF1/Cip1}′P′ or p21[6KR]^{WAF1/Cip1} is expressed under the control of this promoter.
Use of the p21[6KR]^{WAF1/Cip1} protein which 6 lysine residues of the p21^{WAF1/Cip1} native protein were substituted with arginine residues, thus suppressing the internal ubiquitination sites, guaranties in addition that the selected molecules of the invention do not interfere with the other components of the ubiquitin-proteasome pathway such as the ubiquitin ligases, the ubiquitin-conjugating enzymes and the ubiquitin-activating enzyme.
recombinant yeast strain stability: the methods for integrating in a chosen site of a yeast chromosome and for substituting genes in a targeted way enable to prepare recombinant yeast strains expressing the fusion protein containing either p21^{WAF1/Cip1}, or p21[6KR]^{WAF1/Cip1} from the yeast chromosomes. These recombinant yeast strains are therefore genetically stable and may be duplicated and kept indefinitely.
Speed of growth and screening: yeast is a microorganism growing at a high speed and providing a high yield. In particular, the screening method of the invention is preferably performed by culturing the yeast cells in a complete culture medium, in which the yeast cell growth is particularly fast and the yield particularly high, thus making it possible to obtain a great amount of recombinant yeast cells for simultaneously carrying out a plurality of screening tests.
Low cost: yeast is a microorganism which culture, storage and characterization are not expensive,
Automation of the screening method of the invention: yeast is a microorganism which culture, made in a small volume of medium, at low temperature, under usual atmosphere, in air, is very especially adapted to automation (robotization) of the screening methods.

The screening methods of the invention are especially useful to select and characterize active agents such as antineoplastic agents, anti-inflammatory agents, antiviral agents, agents against fungal and bacterial infections, or agents against diseases of the central nervous system.

Without being limited thereto, the present invention will be illustrated hereafter by means of the following figures and examples.

### DESCRIPTION OF THE FIGURES

FIG. 1 shows the maps of the recombinant plasmids encoding a p21-detectable protein fusion protein. FIG. 1A shows a schema of the pCSYAQ6-p21 plasmid containing an expression cassette encoding the mAG-p21 fusion protein. FIG. 1B shows a schema of the pCSYAQ6-p21-6KR plasmid containing an expression cassette encoding the mAG-p21[6KR] fusion protein.

FIG. 2 shows the nucleotide sequence and the amino acid sequence of the p21[6KR]^{WAF1/Cip1} protein.

FIG. 3 shows immunoblot photographs (“Western Blotting”) illustrating the degradation of the fusion protein comprising p21^{WAF1/Cip1} or p21[6KR]^{WAF1/Cip1} fused with mAG, in the presence or in the absence of a proteasome inhibitor, the MG132 compound. The results are given for the following recombinant yeast strains: CYS343 (FIG. 2A) and CYS344 (FIG. 2B).

FIG. 4 shows the results of an epifluorescence microscopic analysis, of the degradation of the mAG-p21[6KR]^{WAF1/Cip1} fusion protein in yeast cells of the CYS344 recombinant strain. FIG. 3A shows the epifluorescence results obtained with the cells of the CYS344 strain cultured in the presence of glucose and at time 0, 60 minutes, 120 minutes and 180 minutes (from the upper part to the lower part of the figure) after stopping the fusion protein expression induction. FIG. 3B shows the epifluorescence results obtained with the cells of the CYS344 strain cultured in the presence of glucose and at time 0, 60 minutes, 120 minutes and 180 minutes (from the upper part to the lower part of the figure) after stopping the fusion protein expression induction.

FIG. 5 shows the quantification, by flow cytometry analysis of the yeast cells of each CYS343 and CYS344 strains, of the fluorescence signal emitted by these cells, during the culture period, respectively (i) during the production induction of the fusion protein in the presence of galactose and (ii) in the presence of glucose after stopping the fusion protein production. The yeast cells were cultured in the presence or in the absence of the MG132 proteasome inhibitor. FIG. 4A shows the flow cytometric quantification (FACS) of the fluorescence emitted by the mAG-p21^{WAF1/Cip1} fusion protein produced by the CYS343 strain, in the presence and in the absence of the MG132 proteasome inhibitor. FIG. 4B shows a flow cytometric quantification (FACS) of the fluorescence emitted by the mAG-p21[6KR]^{WAF1/Cip1} fusion protein produced by the CYS344 strain, in the presence and in the absence of the MG132 proteasome inhibitor.

### EXAMPLES

### Examples 1 to 3

### A. Material and Methods

### A.1. Summary of the Polynucleotide Sequences Used

In the descriptions which follow,

the sequence of the p21^{WAF1/Cip1} protein is the one deposited in the EMBL database under the access number BC001935.1

The sequence of the p21[6KR]^{WAF1/Cip1} protein is the one deposited in the EMBL database under the access number BC001935.1 the 6 lysine residues of which were converted into Arginine residues.

The sequence of the mAG (monomeric Azami-Green) gene derived from Galaxeidae coral encoding the fluorescent protein hereafter referred to as mAG is the one deposited in the GenBank™/EBI Data Bank under the access number AB108447.

The sequence of the pRS306 plasmid is the one deposited in the EMBL database, identifier “PRS306”, access number 003438.

The sequence of the GAL1 gene promoter from the yeast S. cerevisiae used in the descriptions which follow is fully contained in the sequence deposited in the EMBL database, identifier “SCGAL10”, access number K02115.

The sequence of the GAL1 gene promoter from the yeast S. cerevisiae, that may be used in the invention includes the one described by Johnston and Davis (Mol. Cell. Biol. (1984) 4 (8): 1440-1448).

The sequence of the MET3 gene promoter from the yeast S. cerevisiae, that may be used in the invention includes the one described by Cherest and al. (Mol. Gen. Genet. (1987) 210 (2): 307-313).

The sequence of the MET25 gene promoter from the yeast S. cerevisiae, that may be used in the invention includes the one described in Kerjan and al. (Nucleic Acids Res. (1986) 14(20): 7861-7871).

The sequence of the PHO5 gene promoter from the yeast S. cerevisiae, that may be used in the invention includes the one described by Feldmann and al. (EMBO J. (1994) 13(24): 5795-5809).

The sequence of the THI4 gene promoter from the yeast S. cerevisiae, that may be used in the invention includes the one described in Skala and al., Yeast (1995) 14:1421-1427.

The sequence of the CUP1 gene promoter from the yeast S. cerevisiae, that may be used in the invention includes the one described by Karin and al. (PNAS (1984) 81(2): 337-341).

### A.2. Conventions Used

Descriptions are effected by using the nomenclature and the typographic rules that are commonly used in the community of the biologists specialized in the yeast Saccharomyces cerevisiae.
The wild-type name of a gene is indicated by capital italic letters; example: GAL1.
The mutant form name of a gene is indicated by lowercase italic letters, the allele number if any known is given following a dash; example cup1-1.
The inactivated allele name of a gene is indicated by lowercase letters followed with two points×2, then with the name of the inactivating gene ex ppr1::TRP1 (in this example the ppr1 inactivated gene was interrupted by the TRP1 active gene).
   Alternatively, the name of an inactivated gene may be indicated by the “delta” symbol; example gal4Δ
The name of the protein is the one of the gene which encodes the same given in lower case letters, except the first letter which is in caps; for ex. Gal4 (alternatively, the same symbol may be used, followed with a p; example Gal4p).
   A.3. Opening Remarks about the Plasmid Construction

The whole plasmids were constructed by using traditional molecular biology techniques according to protocols described by Sambrook and al. (in Molecular Cloning, Laboratory Manual, 2″ edition, (1989), Cold Spring Harbor, N.Y.) and Ausubel and al., (in Current Protocols in Molecular Biology, (1990-2004), John Wiley and Sons Inc, N.Y,), Molecular cloning, DNA plasmid propagation and production were effected in the Escherichia coli DH10B strain.

### Example 1

### Construction of the Plasmids Enabling the Expression in the Yeast of the mAG-p21^{WAF1/Cip1} and mAG-p21[K6R]^{WAF1/Cip1} Fusion Proteins

The following plasmids enable the expression, in the yeast Saccharomyces cerevisiae, of derivatives of the p21^{WAF1/Cip1} human protein or of the human mutant protein p21[6KR]^{WAF1/Cip1} fused to the mAG fluorescent protein derived from the Galaxeidae coral.

A 614-base pair fragment (bp) corresponding to the GALS gene promoter (pGAL1) from the yeast Saccharomyces cerevisiae was amplified by a polymerase chain reaction (PCR) from genomic DNA of a S. cerevisiae wild strain, X2180-1A, by using the following oligonucleotides:

(i) “pGAL1(Asp)Forw” of sequence
[SEQ ID N^{o} 10]
5′-GCTGGGTACCTTAATAATCATATTACATGGCATTA-3′,
and
(ii) “pGAL1(Xho)Rev” of sequence
[SEQ ID N^{o} 11]
5′-CGACCTCGAGTATAGTTTTTTCTCCTTGACGTTAA-3′.

The fragment obtained was digested by the Asp718I and XhoI restriction enzymes and inserted into the S. cerevisiae-E. coli pRS306 shuttle plasmid previously digested by the Asp7181 and XhoI enzymes, providing the pRS306-pGAL1 vector.

A 678-base pair fragment (bp) corresponding to a variant of the gene encoding the monomeric fluorescent protein Azami Green (mAG) derived from Galaxeidae coral, which sequence was optimized for the yeast expression, was obtained by enzymatic digestion by the XhoI and EcoRI restriction enzymes from the pPCR-Script-mAG vector. The obtained fragment was inserted into the pRS306-pGAL1 plasmid previously digested by the XhoI and EcoRI enzymes, providing the pRS306-pGAL1-mAG vector.

A 340-base pair fragment (bp) corresponding to the terminator of the ADH1 gene (tADH1) from the yeast Saccharomyces cerevisiae was amplified by a polymerase chain reaction (PCR) from genomic DNA of a wild strain of S. cerevisiae X2180-1A, by using the following oligonucleotides:

(i) “TermADH1(NotIBstXI)5′” of sequence
[SEQ ID N^{o} 12]
5′-GGCGGCGGCCGCCACCGCGGTGGGCGAATTTCTTATGATTTA
TG-3′
and
(ii) “TermADH1(SacI)3′” of sequence
[SEQ ID N^{o} 13]
5′-GGCGGAGCTCTGGAAGAACGATTACAACAG-3′.

The obtained fragment was digested by the SacI and NotI restriction enzymes and inserted into the pRS306-pGAL1-mAG plasmid previously digested by the SacI and NotI enzymes, providing the pCSYAQ6 vector.

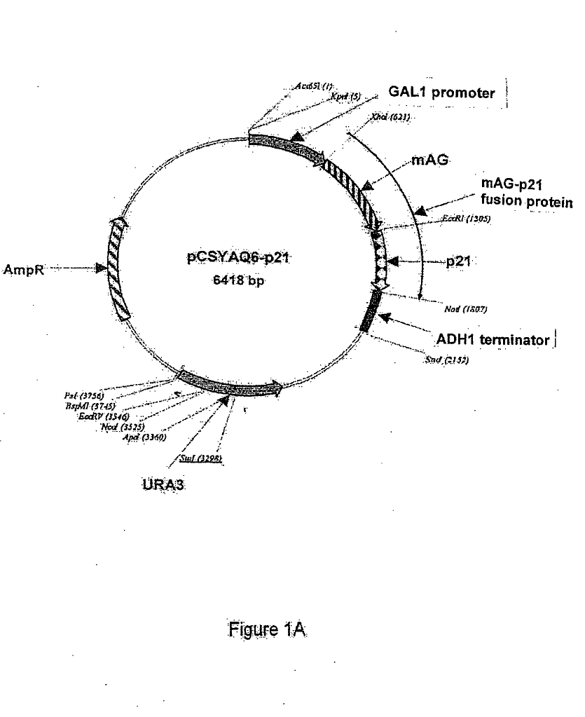

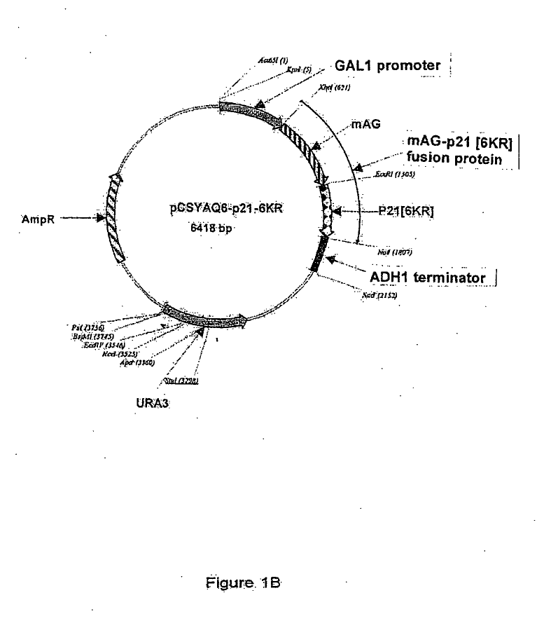

The gene encoding the p21^{WAF1/Cip1} protein which sequence was optimized for the yeast expression was purified from the pPCR-Script-p21WAF1/Cip1[wt] plasmid by digestion by the EcoRI and NotI restriction enzymes. The fragment was cloned in the pCSYAQ6 plasmid prepared by a digestion by the EcoRI and NotI restriction enzymes. The resulting vector was called pCSYAQ6-p21[wt], which diagram is shown on FIG. 1A.

The gene encoding the p21[6KR]^{WAF1/Cip1} protein, which sequence was optimized for the yeast expression, was purified from the pPCR-Script-p21WAF1/Cip1[6KR] plasmid by digestion by the EcoRI and NotI restriction enzymes. The fragment was cloned in the pCSYAQ6 plasmid prepared by a digestion by the EcoRI and NotI restriction enzymes. The resulting vector was called pCSYAQ6-p21[6KR], which diagram is shown on FIG. 1B.

### Construction of the Yeast CYS343 and CYS344 Strains

To obtain the CYS343 yeast strain, the pCSYAQ6-p21[wt] plasmid described hereabove was linearized by using the Stu1 enzyme. The Stu1 enzyme cleaved the pCSYAQ6-p211[wt] plasmid at a unique position located in the sequence of the URA3 gene. The linearized pCSYAQ6-p21[wt] plasmid did integrate to the URA3 locus located on the chromosome 5 left arm of the yeast Saccharomyces cerevisiae.

To obtain the CYS344 yeast strain, the pCSYAQ6-p21[6KR] plasmid described hereabove was linearized by using the Stu1 enzyme. The Stu1 enzyme cleaved the pCSYAQ6-p21[6KR] plasmid at a unique position located in the sequence of the URA3 gene. The linearized pCSYAQ6-p21[6KR] plasmid did integrate to the locus URA3 located on the chromosome 5 left arm of the yeast Saccharomyces cerevisiae.

The CYS343 and CYS344 yeast strains were obtained as described hereabove, in accordance with the general protocol described by Rohthstein (1991).

The respective genotypes of the yeast strains of the invention are given in Table 1 hereunder.

TABLE 1
Strain genotypes of the yeast Saccharomyces cerevisiae
prepared for the purposes of the present invention.

| Strain | Genotype |
|---|---|
| CYS262 | MATa, his3, leu2, ura3, trp1, pdr1Δ, pdr3Δ |
| CYS343 | Mat-a, his3, leu2, trp1, ura3::pGAL1-mAG-p21[wt]::URA3, |
| | pdr1Δ, pdr3Δ |
| CYS344 | Mat-a, his3, leu2, trp1, ura3::pGAL1-mAG-p21[6KR]::URA3, |
| | pdr1Δ, pdr3Δ |

### Examples 2 to 6

### Development of the Screening Method of the Invention

### Example 2

### Nucleotide and Protein Sequences of the p21[6KR]^{WAF1/Cip1} Human Factor Mutant Form Expressed in the Yeast Cells

The amino acid sequence encoding the fusion protein comprising the protein p21[6KR]^{WAF1/Cip1} consists in the polypeptide of SEQ ID No 4 which may be encoded by the polynucleotide of SEQ ID No 6, the codon identity of which was specifically adapted to the optimal expression in the yeast cells.

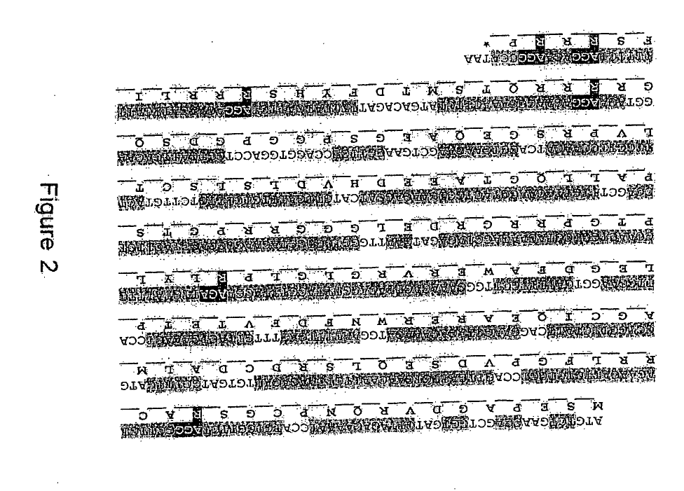

The nucleotide sequence and the amino acid sequence of the p21[6KR]^{WAF1/Cip1} protein are given on FIG. 2.

On FIG. 2, the 6 lysine residues (K) which were substituted with arginine residues (R) are highlighted in black, as well as the corresponding codons. The codons optimized for the expression of the p21[6KR]^{WAF1/Cip1} factor in the yeast Saccharomyces cerevisiae are highlighted in grey.

### Example 3

### Cellular Proteasome-Mediated Degradation of the p21^{WAF1/Cip1} and p21[6KR]^{WAF1/Cip1} Human Factors Expressed in the Yeast Cells (Immunoblotting Results)

The degradation of the fusion proteins comprising p21^{WAF1/Cip1} or p21[6KR]^{WAF1/Cip1} fused with mAG by the proteasome of the recombinant yeast cells of CYS 344 and CYS 343 strains was controlled by an immunoblotting technique, in the presence or in the absence of a proteasome inhibitor, the Mg132 compound.

### A. Material and Methods

All the yeast strains used were cultured and analyzed in a similar way.

The cells were cultured in minimal medium in the presence of galactose as a carbon source for 120 minutes.

At the end of these 120 minutes, 2% glucose was added to the culture, in the presence and in the absence of 50 μM of a known proteasome inhibitor, the Mg 132.

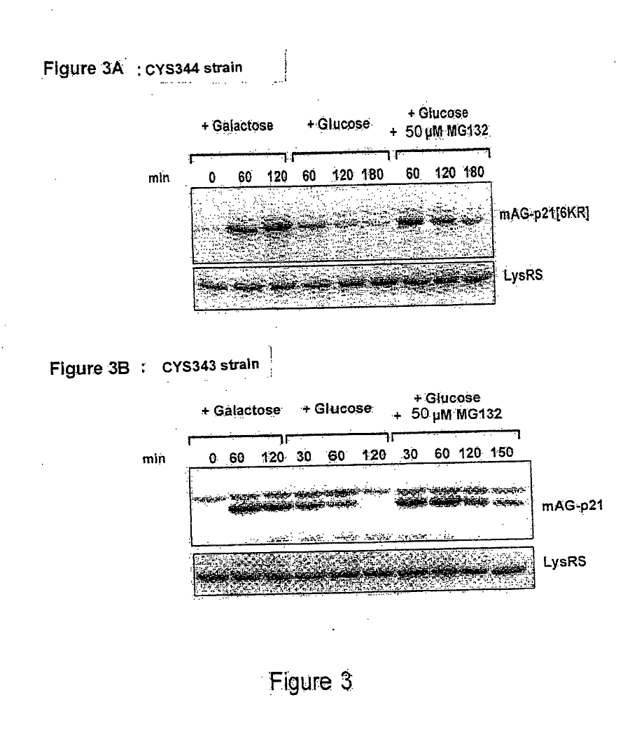

The results are given on FIG. 3 (FIG. 3A and FIG. 3B). FIG. 3A illustrates the results obtained with the CYS344 recombinant yeast strain.

Total proteins were prepared before adding galactose (0), 60 and 120 minutes following the addition of galactose (+galactose 0, 60, 120) and 30, 60 and 120 minutes (+glucose, 30, 60, 120) following the addition of glucose.

FIG. 3B illustrates the results obtained with the CYS343 recombinant yeast strain. Total proteins were prepared before adding galactose (0), 60 and 120 minutes following the addition of galactose (+galactose 0, 60, 120) and 30, 60, 120 and 150 minutes (+glucose, 30, 60, 120, 150) following the addition of glucose.

These proteins were analyzed by immunoblotting technique (“Western blotting”) using an antibody that recognized the p21^{WAF1/Cip1} part of the fusion proteins (noted “mAG-p21^{WAF1/Cip1}” or “mAG-p21[6KR]^{WAF1/Cip1}” on FIG. 3).

The presence of MG132 in the culture is indicated by “+MG132” on FIG. 3. A control of the amount of proteins deposited in each well is carried out by analyzing the same proteins with antibodies that do recognize the yeast Lysyl-RNAt-synthase (noted “LysRS” on FIG. 3). Gel immunoblots (“Western blotting”) were revealed by anti-p21 antibodies and anti-Lys-p21 antibodies

### B. Results

The results are given for the following recombinant yeast strains: CYS343 (FIG. 3A) and CYS344 (FIG. 3B).

The results illustrated on FIG. 3A show by means of a biochemical analysis using Western blotting, the degradation of the mAG-p21[6KR]^{WAF1/Cip1} fusion protein in which the 6 lysine residues of the p21^{WAF1/Cip1} factor were substituted with arginine residues.

The results illustrated on FIG. 3B show by means of a biochemical analysis using Western blotting, the degradation of the mAG-p21 [wt]^{WAF1/Cip1} fusion protein in yeast cells.

On FIGS. 3A and 3B, it can be observed that the presence of galactose (noted “+Galactose” on FIG. 3) during the whole culture time period caused a continuous induction of the production of the p21[6KR]^{WAF1/Cip1}-containing (FIG. 3A) or p21[wt]^{WAF1/Cip1}-containing fusion protein (FIG. 3B), which did counteract the proteasome-mediated fusion protein degradation which did simultaneously occur in the yeast cells.

On FIGS. 3A and 3B, it can be observed that the cell culture in the presence of glucose (noted “+Glucose” on FIG. 3), because the production of the fusion protein is no more induced, did enable to visualise the degradation of the fusion protein by the proteasome.

On FIG. 3A and FIG. 3B, it can be observed that the cell culture in the presence of glucose and a proteasome inhibitor, the MG132 compound (noted “+Glucose+50 μM MG132” on FIG. 3) does enable to inhibit the degradation of the fusion protein which was observed in the absence of the MG132 compound.

### Example 4

### Degradation of the mAG-p21[6KR]^{WAF1/Cip1} Protein (Epifluorescence Microscopy)

Example 4 shows the results of an epifiuorescence microscopic analysis of the mAG-p21[6KR]^{WAF1/Cip1} fusion protein degradation in yeast cells of the CYS344 recombinant strain.

### A. Material and Methods

The yeast cells of the CYS344 strain were cultured in minimal medium in the presence of galactose as a carbon source for 120 minutes.

At the end of these 120 minutes, 2% glucose was added to the culture, in the presence and in the absence of 50 μM of a known proteasome inhibitor, the MG132.

The cells were observed using an epifluorescence microscope (fluorescent microscope Nikon Eclipse fitted with an Omega XF116 filter). All the pictures were saved on a Hamamastu® camera using similar settings, then analyzed with the LUCIA G software, immediately before adding glucose (0 minute), and 60, 120 and 180 minutes (60, 120, 180 minutes) following the addition of glucose. The presence of MG132 in the culture is indicated by MG132″.

### B. Results

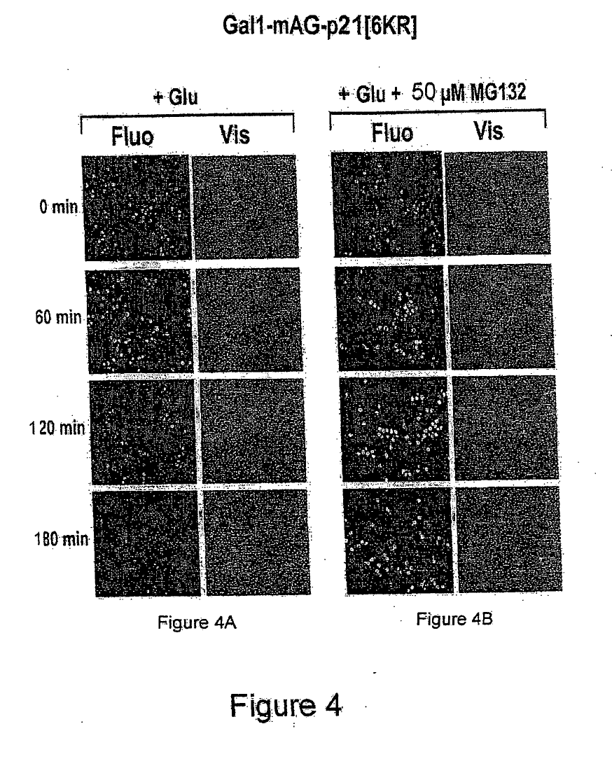

The results are illustrated on FIG. 4 (FIG. 4A and FIG. 4B). FIG. 4A shows the epifluorescence results obtained with the cells of the CYS344 strain cultured in the presence of glucose and at time 0, 60 minutes, 120 minutes and 180 minutes (from the upper part to the lower part of the figure) after stopping the fusion protein expression induction. FIG. 4B shows the epifluorescence results obtained with the cells of the CYS344 strain cultured in the presence of glucose and at time 0, 60 minutes, 120 minutes and 180 minutes (from the upper part to the lower part of the figure) after stopping the fusion protein expression induction.

On FIG. 4A and FIG. 4B, the column on the left shows the fluorescence microscopy photographs enabling to localize in the cells the expression of the mAG-p21[6KR]^{WAF1/Cip1} fusion protein.

On FIG. 4A and FIG. 4B, the column on the right provides a visualization of the same cells, under visible light.

On FIG. 4A, a progressive decrease in the fluorescence signal was observed versus culture time, as the proteasome-mediated fusion protein degradation was progressing (column on the left), whereas the cell density in the observed field remained unchanged (column on the right).

On FIG. 4B, it could be observed that the fluorescence signal intensity versus culture time was maintained (column on the left), whereas the cell density in the observed field remained unchanged (column on the right), which shows the inhibition of the fusion protein degradation activity of the proteasome, which inhibition is induced by the MG132 inhibitor.

### Example 5

### Degradation of the mAG-p21[6KR]^{WAF1/Cip1} Protein (Flow Cytometric Quantification)

Example 5 shows a quantification by flow cytometry analysis of the yeast cells from each of the CYS343 and CYS344 strains, of the fluorescence signal emitted by these cells, during the culture period, respectively (i) during the production induction of the fusion protein in the presence of galactose and (ii) in the presence of glucose after stopping the fusion protein production. The yeast cells were cultured in the presence or in the absence of the MG132 proteasome inhibitor.

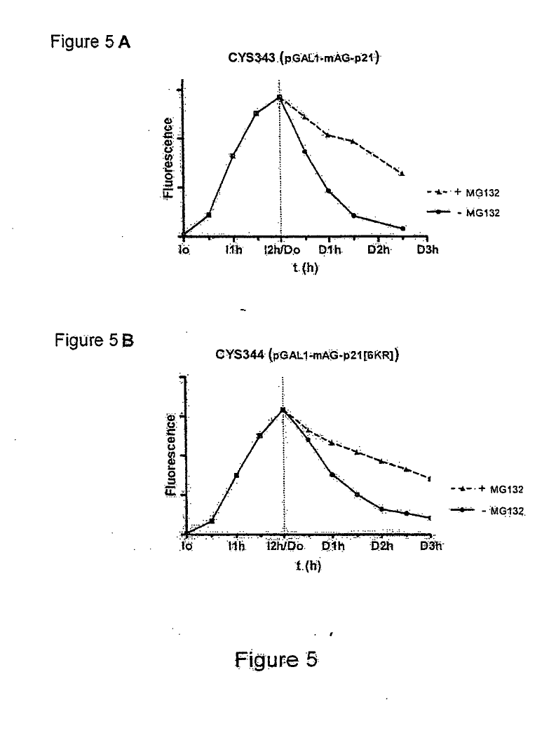

The results are illustrated with the curves shown on FIG. 5.

FIG. 5A shows a flow cytometric quantification (FACS) of the fluorescence emitted by the mAG-p21^{WAF1/Cip1} fusion protein produced by the CYS343 strain, in the presence and in the absence of the MG132 proteasome inhibitor.

FIG. 5B shows a flow cytometric quantification (FACS) of the fluorescence emitted by the mAG-p21[6KR]^{WAF1/Cip1} fusion protein produced by the CYS344 strain, in the presence and in the absence of the MG132 proteasome inhibitor.

### A. Material and Methods

All the cells were cultured in minimal medium in the presence of galactose as a carbon source for 120 minutes.

At the end of these 120 minutes, 2% glucose was added to the culture, in the presence and in the absence of 50 μM of a known proteasome inhibitor, the MG132.

The fluorescence emitted by the cells was quantified using a FacsCalibur flow cytometer (Beckton-Dickinson), immediately before adding galactose (I0), 1 and 2 hour(s) following the addition of galactose (I1 and I2) and 1, 2 and 3 hours (D1, D2 and D3) following the addition of glucose. The presence of Mg132 in the culture is indicated by “+MG132”. The fluorescence is expressed in arbitrary units.

### B. Results

The results of FIGS. 5A and 5B show that the fusion protein is actively produced by the yeast CYS343 and CYS344 strains, when the cells are cultured in the presence of galactose (time Io, I1h, I2h/D0).

The results of FIGS. 5A and 5B show that the fusion protein, which accumulated in the cells in the presence of galactose, is gradually degraded during the culture period in the absence of galactose and in the presence of glucose, in the absence of MG132 compound (Time D1 h, D2h, D3h).

The results of FIGS. 5A and 5B show that, in the presence of the MG132 proteasome inhibitor, the degradation of the fusion protein is strongly inhibited (Time D1 h, D2h, D3h).

### Example 6

### Localization in the Yeast Cells of the mAG-p21[6KR]^{WAF1/Cip1} Fusion Proteins

In example 6, the localization in the CYS344 strain yeast cells of the mAG-p21[6KR]^{WAF1/Cip1} protein was determined.

Yeast cells of the CYS344 strain comprising a polynucleotide enabling the expression of the mAG-p21[6KR]^{WAF1/Cip1} fusion protein under the control of the GAL1 promoter were cultured in the presence of 2% galactose for 2 hours and were then observed using a fluorescence microscope.

The nucleus position was revealed by using a nucleus-specific coloured indicator, the Hoechst 333-42 dye.

Light microscopy photographs were taken, as well as fluorescence microscopy photographs in which cell nuclear DNA was stained with the Hoechst 333-42 dye (pictures not shown).

The light microscopy photographs and the fluorescence microscopy photographs were surimposed (not shown).

A colocalization of the cell nuclei and of the mAG-p21[6KR] fusion protein^{WAF1/Cip1} was observed.

### REFERENCE WORK

Adams (Nature Reviews Cancer (2004) 4:349-360)
Adams and Anderson (Cancer Res. (2001) 61:3071-3076)
Ausubel and al. (in Current Protocols in Molecular Biology, (1990-2004), John Wiley and Sons Inc, N.Y.).
Bailis and al., (Genetics (1990), 126:535-547)
Bendjennat and al. (Cell (2003) 114: 599-610)
Bennetzen and Hall. (J. Biol. Chem. (1982) 257: 3018-3025).
Beverly and al., (Clin. Cancer Res. (1999) 5:2638-2645)
Bornstein and al., (J. Biol. Chem. (2003) 278:25752-25757)
Burger and Seth (Eur J. Cancer. (2004) 40:2217-2229)
Coux and al., (Annu Rev Biochem. (1996) 65:801-47)
Johnston and Davis (Mol. Cell. Biol. (1984) 4: 1440-1448).
Glickman and Ciechanover (Physiol Rev. (2002) 82:373-428)
Glickman and Coux (Curr. Protoc. Prot Sci (2001) p21.5.1-21.5.17)
Glickman and al. (Mol. Cell. Biol. (1998) 18: 3149-3162
Glickman and al. (In: Proteasomes: The World of Regulatory Proteolysis, edited by Hilt W and Wolf D H. Georgetown, Tex.: Landes Bioscience, (2000), p. 71-90)
Gurskaya and al., (Biochem. J. (2003) 373:403-408)
Hideshima and al. (Cancer Res. (2001) 61:3071-3076)
Hipp and al. (Mol Cell Biol. (2005) 25:3483-3491.
Holzl and al. (J. Cell Biol. (2000) 150: 119-130)
Karasawa and al. (J. Biol. Chem. (2003) 278:34167-34171)
Karin and al, (PNAS (1984) 81: 337-341).
Kerjan and al. (Nucleic Acids Res. (1986) 14: 7861-7871).
Kriwacki and al., (Proc. Natl. Acad. Sci. U.S.A. (1996) 93:11504-11509)
Leggett and al. (Mol. Cell. (2002) 10:495-507)
Liu and al, (Science (2003) 299:408-411)
Matz and al. (Nature Biotech. (1999) 17:969-973)
Pei and al. (Leukemia (2003) 17:2036-2045)
Rothstein, in Methods in Enzymology, (1991), 194:281-301
Sambrook and al. (in Molecular Cloning, Laboratory Manual, 2^{nd} edition, (1989), Cold Spring Harbor, N.Y.)
Schiestl and al. (Curr. Genet. (1989), 16:339-346
Schmidt and al., (Nature Struc. Mol. Biol. (2005) 12:294-303)
Shagin and al, (Mal. Biol. Evol. (2004) 21:841-850)
Sherman and al., in Methods in Yeast Genetics: a Laboratory Manual, (1979), Cold Spring Harbor, N.Y.
Sikorski and Hieter (Genetics (1989) 122: 19-27).
Ustrell and al. (Embo J. (2002) 21:3516-3525)
Wojcik and al. (Eur. J. Cell. Biol. (1998) 77:151-160)
Zhang and al., (J. Biol. Chem. (2004) 279: 16000-16006)

TABLE 2
Sequences

| SEQ ID N° | Type | Description |
|---|---|---|
| 1 | Polypeptide | p21^{WAF1/Cip1} protein |
| 2 | Polypeptide | p21[6KR]^{WAF1/Cip1} protein |
| 3 | Polypeptide | mAG - p21^{WAF1/Cip1} fusion protein |
| 4 | Polypeptide | mAG - p21[6KR]^{WAF1/Cip1} fusion protein |
| 5 | Nucleic acid | mAG - p21^{WAF1/Cip1} fusion protein |
| 6 | Nucleic acid | mAG - p21[6KR]^{WAF1/Cip1} fusion protein |
| 7 | Polypeptide | HA antigen |
| 8 | Polypeptide | FLAG antigen (monomer) |
| 9 | Polypeptide | FLAG antigen (trimer) |
| 10-13 | Nucleic acid | Primers |

1. An in cellulo method for the screening of proteasome activity-modulating agents, said method comprising the following steps of:
a) bringing into contact a candidate agent to be tested with recombinant yeast cells expressing in their nucleus a fusion protein comprising:
(i) a p21 polypeptide selected from the p21^{WAF1/Cip1} polypeptide and the p21[6KR]^{WAF1/Cip1} polypeptide; and
(ii) at least one detectable protein;
b) quantifying said first detectable protein in the yeast cells, at the end of at least one predetermined time interval after bringing into contact the candidate agent with said cells;
c) comparing the value obtained at the step (b) with a control value obtained when the step (a) is performed in the absence of the candidate agent.

2. A method according to claim 1, wherein step (a) comprises the following steps of:
(a1) culturing the yeast cells which do express in their nucleus said fusion protein comprising the p21 polypeptide and at least one detectable protein;
(a2) stopping the expression by the yeast cells of said fusion protein comprising the p21 polypeptide and at least one detectable protein;
(a3) bringing into contact the yeast cells obtained at the end of the step (a2) with the candidate agent to be tested.

3. A method according to claim 1, wherein the detectable protein comprised in the p21 polypeptide-containing fusion protein is selected from an antigen, a fluorescent protein and a protein having an enzymatic activity.

4. A method according to claim 3, wherein the detectable protein consists of a fluorescent protein selected from the mAG protein or a derivative thereof, and the GFP protein or a derivative thereof.

5. A method according to claim 3, wherein the detectable protein consists of a protein having an enzymatic activity selected from the luciferase and the β-lactamase.

6. A method according to claim 3, wherein the detectable protein consists of an antigen selected from the Ha peptide and the Flag peptide.

7. A method according to claim 1, wherein the protein comprising the p21^{WAF1/Cip1} polypeptide consists in the protein of SEQ ID No 3.

8. A method according to claim 1, wherein the protein comprising the p21[6KR]^{WAF1/Cip1} polypeptide consists in the protein of SEQ ID No 4.

9. A method according to claim 1, wherein at step (b), when the first detectable protein is an antigen, said first detectable protein is quantified by detecting the complexes formed between said protein and antibodies recognizing the same.

10. A method according to claim 1, wherein at step (b), when the first detectable protein is a fluorescent protein, said detectable protein is quantified by measuring the fluorescence signal emitted by said protein.

11. A method according to claim 1, wherein at step (b), when the first detectable protein is a protein having an enzymatic activity, said detectable protein is quantified by measuring the substrate amount transformed by said protein.

12. A method according to claim 1, wherein the recombinant yeast cells are transformed with a polynucleotide which comprises:
(a) an open reading frame encoding (i) the fusion protein comprising the p21 polypeptide (p21^{WAF1/Cip1} or p21[6KR]^{WAF1/Cip1}) and (ii) a detectable protein, and
(b) a regulating sequence which is functional in yeast cells and controls the expression of said open reading frame.

13. A method according to claim 12, wherein the regulating sequence contained in the first polynucleotide comprises a promoter which is functional in the yeast cells and which is sensitive to the action of an inducing agent.

14. A method according to claim 13, wherein the promoter sensitive to the action of an inducing agent consists of a repressible promoter, functional in the yeast cells, selected from CUP1, GAL1, GAL10, MET3, MET25, PHO5, and THI4 of the yeast Saccharomyces cerevisiae.

15. A method according to claim 14, wherein said polynucleotide encoding the fusion protein comprises the GAL1 regulating sequence, which activates the expression of the open reading frame encoding said fusion protein.

16. A method according to claim 1, wherein the recombinant yeast cells possess the polynucleotide in a form integrated in the genome thereof.

17. A method according to claim 1, wherein the recombinant yeast cells possess in their genome an inactivated form of one or more gene(s) controlling the expression of transporter proteins integrated in the plasma membrane.

18. A method according to claim 17, wherein the inactivated gene(s) is or are selected from the PDR1 and PDR3 genes.

19. An expression cassette, functional in the yeast cells, comprising an encoding polynucleotide which comprises an open reading frame encoding the fusion protein comprising the p21 polypeptide and at least one detectable protein, and a regulating sequence which is functional in yeast cells and controls the expression of said open reading frame.

20. A functional expression cassette according to claim 19, wherein the p21 polypeptide is selected from the p21^{WAF1/Cip1} polypeptide and the p21[6KR]^{WAF1/Cip1} polypeptide.

21. A functional expression cassette according to claim 20, wherein the p21 polypeptide is selected from the p21^{WAF1/Cip1} polypeptide of SEQ ID No 1 and the p21[6KR]^{WAF1/Cip1} polypeptide of SEQ ID No 2.

22. An expression cassette according to claim 19, wherein the regulating sequence contained in said polynucleotide comprises a promoter which is functional in the yeast cells and which is sensitive to the action of an inducing agent.

23. An expression cassette according to claim 22, wherein the inducible promoter which is functional in the yeast cells is selected from CUP1, GAL1, GAL10, MET3, MET25, PHO5, and TH14 of the yeast Saccharomyces cerevisiae.

24. An expression vector characterized in that it comprises an expression cassette according to claim 19.

25. An expression vector according to claim 24, which is the pCSYAQ6-p21[wt] vector.

26. An expression vector according to claim 24, which is the pCSYAQ6-p21[6KR] vector.

27. A recombinant yeast strain comprising, in a form integrated in the genome thereof, a polynucleotide which comprises (a) an open reading frame encoding the fusion protein comprising a p21 polypeptide and at least one detectable protein, and (b) a regulating sequence which is functional in yeast cells and controls the expression of said open reading frame.

28. A recombinant yeast strain according to claim 27, wherein the p21 polypeptide is selected from the p21^{WAF1/Cip1} polypeptide and the p21[6KR]^{WAF1/Cip1} polypeptide.

29. A recombinant yeast strain according to claim 28, wherein the p21 polypeptide is selected from the p21^{WAF1/Cip1} polypeptide of SEQ ID No 1 and the p21[6KR]^{WAF1/Cip1} polypeptide of SEQ ID No 2.

30. A kit for the screening of proteasome activity-modulating agents comprising an expression vector containing an expression cassette according to claim 19.

31. A kit for the screening of proteasome activity-modulating agents, comprising recombinant yeast cells containing, in a form integrated in the genome thereof, an expression cassette according to claim 19.


SEQUENCE LISTING (14 sequences)

SEQ ID NO: 1 - protein, 164 aa, Homo sapiens
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 2 - protein, 164 aa, Artificial Sequence
  Description of Artificial Sequence Synthetic polypeptide
MSEPAGDVRQNPCGSRACRRLFGPVDSEQLSRDCDALMAGCIQEARERWNFDFVTETPLEGDFAWERVRGLGLPRLYLPTGPRRGRDELGGGRRPGTSPALLQGTAEEDHVDLSLSCTLVPRSGEQAEGSPGGPGDSQGRRRRQTSMTDFYHSRRRLIFSRRRP

SEQ ID NO: 3 - protein, 392 aa, Artificial Sequence
  Description of Artificial Sequence Synthetic polypeptide
MVSVIKPEMKIKLCMRGTVNGHNFVIEGEGKGNPYEGTQILDLNVTEGAPLPFAYDILTTVFQYGNRAFTKYPADIQDYFKQTFPEGYHWERSMTYEDQGICTATSNISMRGDCFFYDIRFDGTNFPPNGPVMQKKTLKWEPSTEKMYVEDGVLKGDVNMRLLLEGGGHYRCDFKTTYKAKKEVRLPDAHKIDHRIEILKHDKDYNKVKLYENAVARYSMLPSQAKEFMSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 4 - protein, 392 aa, Artificial Sequence
  Description of Artificial Sequence Synthetic polypeptide
MVSVIKPEMKIKLCMRGTVNGHNFVIEGEGKGNPYEGTQILDLNVTEGAPLPFAYDILTTVFQYGNRAFTKYPADIQDYFKQTFPEGYHWERSMTYEDQGICTATSNISMRGDCFFYDIRFDGTNFPPNGPVMQKKTLKWEPSTEKMYVEDGVLKGDVNMRLLLEGGGHYRCDFKTTYKAKKEVRLPDAHKIDHRIEILKHDKDYNKVKLYENAVARYSMLPSQAKEFMSEPAGDVRQNPCGSRACRRLFGPVDSEQLSRDCDALMAGCIQEARERWNFDFVTETPLEGDFAWERVRGLGLPRLYLPTGPRRGRDELGGGRRPGTSPALLQGTAEEDHVDLSLSCTLVPRSGEQAEGSPGGPGDSQGRRRRQTSMTDFYHSRRRLIFSRRRP

SEQ ID NO: 5 - DNA, 1179 bp, Artificial Sequence
  Description of Artificial Sequence Synthetic polynucleotide
atggtttcagttattaagccagaaatgaagattaagttgtgtatgagaggtacagttaatggtcataatttcgttattgaaggtgaaggtaaaggtaatccatacgaaggtactcaaattttggatttgaatgttactgaaggtgctccattgccatttgcttacgatattttgactacagtttttcaatacggtaatagagcttttactaagtacccagcagatattcaagattactttaagcaaacttttccagaaggttaccattgggaaagatcaatgacttacgaagatcaaggaatttgtactgctacttctaatatttctatgagaggagattgttttttctacgatattagattcgatggtactaattttccaccaaatggtccagttatgcaaaaaaagactttgaagtgggaaccatctacagaaaagatgtacgttgaagatggtgttttgaagggtgatgttaatatgagattgttgttggaaggtggtggtcattacagatgtgattttaagactacttacaaggctaaaaaggaagttagattgccagatgctcataagatagatcatagaatagaaattttgaagcacgataaggattacaacaaggttaagttatacgaaaatgctgttgcaagatactcaatgttgccatctcaagctaaggaattcatgtctgaaccagctggtgatgttagacaaaatccatgtggttctaaggcttgtagaagattgtttggtccagttgattctgaacaattgtcaagagattgtgatgctttgatggctggttgtattcaggaagctagagaaagatggaattttgattttgttactgaaactccattggaaggtgattttgcttgggaaagagttagaggtttaggtttaccaaaattgtatttgccaactggtcctagaagaggtagagatgaattgggtggtggtagaaggccaggtacttctccagctttgttgcaaggtactgctgaagaggatcatgttgatttgtctttgtcttgtactttggttccaagatcaggtgaacaagctgaaggttctccaggtggacctggtgattcacaaggtagaaagagaaggcaaacttctatgacagatttttatcattctaagagaagattgattttttctaagagaaaaccataa

SEQ ID NO: 6 - DNA, 1179 bp, Artificial Sequence
  Description of Artificial Sequence Synthetic polynucleotide
atggtttcagttattaagccagaaatgaagattaagttgtgtatgagaggtacagttaatggtcataatttcgttattgaaggtgaaggtaaaggtaatccatacgaaggtactcaaattttggatttgaatgttactgaaggtgctccattgccatttgcttacgatattttgactacagtttttcaatacggtaatagagcttttactaagtacccagcagatattcaagattactttaagcaaacttttccagaaggttaccattgggaaagatcaatgacttacgaagatcaaggaatttgtactgctacttctaatatttctatgagaggagattgttttttctacgatattagattcgatggtactaattttccaccaaatggtccagttatgcaaaaaaagactttgaagtgggaaccatctacagaaaagatgtacgttgaagatggtgttttgaagggtgatgttaatatgagattgttgttggaaggtggtggtcattacagatgtgattttaagactacttacaaggctaaaaaggaagttagattgccagatgctcataagatagatcatagaatagaaattttgaagcacgataaggattacaacaaggttaagttatacgaaaatgctgttgcaagatactcaatgttgccatctcaagctaaggaattcatgtctgaaccagctggtgatgttagacaaaatccatgtggttctagggcttgtagaagattgtttggtccagttgattctgaacaattgtcaagagattgtgatgctttgatggctggttgtattcaggaagctagagaaagatggaattttgattttgttactgaaactccattggaaggtgattttgcttgggaaagagttagaggtttaggtttaccaagattgtatttgccaactggtcctagaagaggtagagatgaattgggtggtggtagaaggccaggtacttctccagctttgttgcaaggtactgctgaagaggatcatgttgatttgtctttgtcttgtactttggttccaagatcaggtgaacaagctgaaggttctccaggtggacctggtgattcacaaggtagaaggagaaggcaaacttctatgacagatttttatcattctaggagaagattgattttttctaggagaaggccataa

SEQ ID NO: 7 - protein, 9 aa, Artificial Sequence
  Description of Artificial Sequence Synthetic peptide
YPYDVPDYA

SEQ ID NO: 8 - protein, 8 aa, Artificial Sequence
  Description of Artificial Sequence Synthetic peptide
DYKDDDDK

SEQ ID NO: 9 - protein, 23 aa, Artificial Sequence
  Description of Artificial Sequence Synthetic peptide
MDYKDHDGDYKDHDIDYKDDDDK

SEQ ID NO: 10 - DNA, 35 bp, Artificial Sequence
  Description of Artificial Sequence Synthetic oligonucleotide
gctgggtaccttaataatcatattacatggcatta

SEQ ID NO: 11 - DNA, 35 bp, Artificial Sequence
  Description of Artificial Sequence Synthetic oligonucleotide
cgacctcgagtatagttttttctccttgacgttaa

SEQ ID NO: 12 - DNA, 44 bp, Artificial Sequence
  Description of Artificial Sequence Synthetic oligonucleotide
ggcggcggccgccaccgcggtgggcgaatttcttatgatttatg

SEQ ID NO: 13 - DNA, 30 bp, Artificial Sequence
  Description of Artificial Sequence Synthetic oligonucleotide
ggcggagctctggaagaacgattacaacag

SEQ ID NO: 14 - DNA, 495 bp, Artificial Sequence
  Description of Artificial Sequence Synthetic polynucleotide
atgtctgaaccagctggtgatgttagacaaaatccatgtggttctagggcttgtagaagattgtttggtccagttgattctgaacaattgtcaagagattgtgatgctttgatgcctggttgtattcaggaagctagagaaagatggaattttgattttgttactgaaactccattggaaggtgattttgcttgggaaagagttagaggtttaggtttaccaagattgtatttgccaactggtcctagaagaggtagagatgaattgggtggtggtagaaggccaggtacttctccagctttgttgcaaggtactgctgaagaggatcatgttgatttgtctttgtcttgtactttggttccaagatcaggtgaacaagctgaaggttctccaggtggacctggtgattcacaaggtagaaggagaaggcaaacttctatgacagatttttatcattctaggagaagattgattttttctaggagaaggccataa